(19) United States
(12) Patent Application Publication
(10) Pub. No.: US 2022/0361473 A1
(43) Pub. Date: Nov. 17, 2022

(54) DECISION SYSTEM FOR CROP EFFICIENCY PRODUCT APPLICATION USING REMOTE SENSING BASED SOIL PARAMETERS

(71) Applicant: BASF Agro Trademarks GmbH, Ludwigshafen am Rhein (DE)

(72) Inventors: Ole JANSSEN, Köln (DE); Fabian Johannes SCHAEFER, Langenfeld (DE); Christian KERKHOFF, Köln (DE); Andreas JOHNEN, Münster (DE)

(21) Appl. No.: 17/622,157

(22) PCT Filed: Jun. 22, 2020
(86) PCT No.: PCT/EP2020/067334

§ 371 Date: Dec. 22, 2021

(30) Foreign Application Priority Data
Jun. 24, 2019 (EP) 19181924.2

(51) Int. Cl. A01M 7/00; G01N 33/24; G01N 33/00; A01B 69/04; G01C 21/00

(52) U.S. Cl. CPC A01M 7/0089; G01N 33/24; G01N 33/0098; A01B 69/008; G01C 21/3826; G01N 2033/245

(57) ABSTRACT

In order to achieve a more effective application of a crop efficiency product, a computer-implemented method is provided for applying a crop efficiency product to at least one crop in a field. The method comprises the steps of collecting remotely-sensed data of the field before an application of the crop efficiency product in the field, determining, based on the collected remotely-sensed data, at least one soil parameter at a plurality of locations in the field, generating, for each of the plurality of locations, a predicted yield response to the application of the crop efficiency product for the at least one crop based on the at least one determined soil parameter and a prediction model, wherein the prediction model is parametrized or trained based on a sample set including a plurality of different values of the at least one soil parameter and associated yield responses for the at least one crop under the application of the crop efficiency product, deciding, for each of the plurality of locations in the field, whether to treat or not based on the predicted yield response, and outputting information indicative of the decision useable to activate at least one treatment device to comply with the decision.

### FIELD OF THE INVENTION

This invention relates generally to crop management, and more specifically to a computer-implemented method for applying a crop efficiency product in a field, to a decision-support system for controlling a treatment device for applying a crop efficiency product in a field, to a treatment device for applying a crop efficiency product in a field, and to a system for applying a crop efficiency product in a field.

### BACKGROUND OF THE INVENTION

At any stage of growth, crops are at risk from pests and diseases. Crop efficiency products have an impact on the crop health and the resulting yield. For example, herbicides kill or stop the growth of unwanted weeds. Fungicides destroy or prevent the growth of disease-causing fungi. However, the yield response to a crop efficiency product may not be stable. In particular, the crop efficiency product on the crop itself may bear a certain stress, which may result in a negative response. Thus, if the application of the crop efficiency product is done properly, a positive return on investment may be seen. On the other hand, if the application of the crop efficiency product is not done properly, the crop efficiency products may result in a negative yield response.

### SUMMARY OF THE INVENTION

There may be a need to provide a method and a device for a more effective application of a crop efficiency product.

The object of the present invention is solved by the subject-matter of the independent claims. Further embodiments and advantages of the invention are incorporated in the dependent claims. The described embodiments similarly pertain to the computer-implemented method for applying a crop efficiency product in a field, to the decision-support system for controlling a treatment device for applying a crop efficiency product in a field, to the treatment device for applying a crop efficiency product in a field, and to the system for applying a crop efficiency product in a field.

A first aspect of the invention relates to a computer-implemented method for applying a crop efficiency product to at least one crop in a field. The method comprises the steps of collecting, by a data interface, remotely-sensed data of the field before an application of the crop efficiency product in the field, determining, by a parameter determination unit, based on the collected remotely-sensed data, at least one soil parameter at a plurality of locations in the field, generating, by a yield prediction unit, for each of the plurality of locations, a predicted yield response to the application of the crop efficiency product for the at least one crop based on the at least one determined soil parameter and a prediction model, wherein the prediction model is parametrized or trained based on a sample set including a plurality of different values of the at least one soil parameter and associated yield responses for the at least one crop under the application of the crop efficiency product, deciding, by a decision unit, for each of the plurality of locations in the field, whether to treat or not based on the predicted yield response and outputting information indicative of the decision useable to activate at least one treatment device to comply with the decision.

Crop efficiency products on the crop itself may bear a certain stress, which results in a negative response, in certain environmental conditions, so that a positive yield response, even more a positive return on investment is only seen, if the positive effect of the crop efficiency product outpaces the penalty. As the soil parameters, such as the soil moisture and the soil temperate, may vary from one spot to another, the performance of the crop efficiency product may also vary from one spot to another. Thus, in some spots the positive effect of the product efficiency product may outpace the penalty, whereas in other spots the application of the crop efficiency product may result in a negative yield response. The spatial variability of the soil parameters is thus a source of uncertainty for the performance of the crop efficiency product across the field. By considering, before the application of the crop efficiency product, the influence of the soil parameters on the effect of the crop efficiency product, it is possible to determine whether to spray or not at respective spots. In this way, it may be avoided to apply the crop efficiency product to some areas, where a negative resulting yield is expected. Additionally, it may be ensured that the crop efficiency product is applied to the areas, where the positive effect of the crop efficiency product, e.g. fungicide, outpaces the penalty and thus a positive return on investment can be seen. Furthermore, this may also reduce the requirements for the crop efficiency product and the possibility of contaminating irrigation channels and ground water.

The term “crop efficiency product” as used herein may also be referred to as crop protection product for pest and disease control. The crop efficiency product may comprise e.g. herbicides, insecticides, fungicides, and bactericides.

The term “remotely-sensed data” as used herein may refer to the data collected using satellite, drone, or radar platforms. Various remote sensing methods may be used in dependence of the parameters to be measured. For example, optical remote sensing may be carried out to make use of e.g. visible, infrared (IR), near infrared (NIR), short-wave infrared, or multispectral sensors to form images of surface of the field by detecting the solar radiation reflected from targets on the ground. Satellite sensors or radars operating at microwaves, both active and passive, may be used for the remote monitoring of the surface of a field.

The term “soil parameter” as used herein may refer to physical and/or chemical properties of soils in a field. The soil parameter may include e.g. pH, electrical conductivity, texture, moisture, temperature, soil organic matter, available nitrogen, phosphorus and/or potassium. As the spatial variability of the soil parameters is a source of uncertainty for the performance of the crop efficiency product, the measurements of one or more soil parameters, together with a prediction model, can generate a predicted yield response to the application of the crop efficiency product. This allows a farmer to determine whether to apply the crop efficiency product at respective locations.

The term “prediction model” as used herein may denote a model that uses mathematical and computational methods to predict an event or outcome. In an example, the prediction model is a trained computational predictive model, such as a machine learning model, which can be trained using “training data” to recognize patterns, classify data, and forecast future events. Field trials may be conducted to obtain the training data for the machine learning model. For example, the crop efficiency product may be applied to a crop in a field exposed to different soil parameter inputs, such as different soil moistures, different soil surface temperatures, and/or other soil parameters that may affect the performance of the crop efficiency product. The corresponding yields obtained from the field trials, together with the different soil parameter inputs, can be used as training data for the machine learning model. In another example, the prediction model is a parametrized mathematical approach that uses an equation-based model to describe the phenomenon of the influence of the soil parameters on the performance of the crop efficiency product. The mathematical model is used to forecast an outcome at some future state or time based upon changes to the model inputs. The sample data from field trials may be used to fit the parameters of a mathematical equation, which is then used to generate a predicted yield response from measured soil parameters.

Each unit may be part of, or include an Application Specific Integrated Circuit (ASIC), an electronic circuit, a processor (shared, dedicated, or group) and/or memory (shared, dedicated, or group) that execute one or more software or firmware programs, a combinational logical circuit, and/or other suitable components that provide the described functionality.

According to an embodiment of the invention, the method further comprises controlling at least one treatment device to comply with the decision based on the outputted information.

For example, the information may be part of configuration data, which may be loaded onto a treatment device and stored in a volatile memory of the treatment device. In operation, the treatment device may load the stored configuration data and processes the configuration data to perform the treatment.

According to an embodiment of the invention, the at least one soil parameter comprises at least one of the following: a soil moisture, preferably measured at a sub-field resolution in a timeframe in days before the application of the crop efficiency product, and/or a soil surface temperature, preferably measured during a particular time period, preferably during winter.

For example, the soil moisture may be measured in a time frame of 0 to 28 days before the prospected application of the crop efficiency product. For example, a sub-field resolution of 100 meter may be used to allow deciding whether it is worth to treat or not at a sub-field level for each management blocks of the field.

Winter conditions define the survival rate of spores from previous season, leading to a better yield response if more spores have survived winter. It is believed that spore survival rate correlates with soil moisture and land temperature during winter.

According to an embodiment of the invention, the soil surface temperature is predicted by whether forecast data.

Instead of collecting in-season data of soil surface temperature, the soil surface temperature may be predicted by using the weather forecast data based on the data from previous seasons.

According to an embodiment of the invention, determining at least one soil parameter at a plurality of locations in the field further comprises determining, based on the collected remotely-sensed data, at least one vegetation parameter, preferably measured at a sub-field level resolution. Generating a predicted yield response to the application of the crop efficiency product further comprises generating, for each of the plurality of locations, a predicted yield response to the application of the crop efficiency product for the at least one crop based on the at least one determined soil parameter, the at least one vegetation parameter, and a prediction model. The prediction model is parametrized or trained based on a sample set including a plurality of different values of the at least one soil parameter, different values of the at least one vegetation parameter, and associated yield responses for the at least one crop under the application of the crop efficiency product.

Examples of the vegetation parameters are standardized precipitation index (SPI), vegetation optical depth (VOD), normalized difference vegetation index (N DVI), and/or enhanced vegetation index (EVI). The inclusion of the vegetation parameter may improve the accuracy of yield response prediction.

According to an embodiment of the invention, deciding, for each of the plurality of locations, whether to treat or not, further comprises evaluating, based on the predicted yield response, whether a treatment i) deteriorates a growth of the at least one crop, ii) does not affect the growth of the at least one crop, or iii) improves the growth of the at least one crop, determining, for each of the plurality of locations, whether the predicted yield response is above a positive reference value, and deciding, for each of the plurality of locations, whether to treat or not based on the determination result.

For example, a negative predicted yield response may indicate that a treatment may deteriorate a growth of the at least one crop. A predicted yield response with zero value may indicate that a treatment does not affect the growth of the at least one crop. A positive predicted yield response may indicate that a treatment may improve the growth of the at least one crop. A treatment may be reasonable when an appropriate yield response may be achieved—that is, a treatment may be reasonable if the predicted yield response is above a positive reference value. On the other hand, if a low yield response is expected, there is no need to apply the crop efficiency product. Such an effort may reduce the requirements for crop efficiency product and improve a positive return on investment. This may also reduce the contamination of irrigation channels and ground water due to the application of the crop efficiency product.

According to an embodiment of the invention, deciding, for each of the plurality of locations, whether to treat or not, further comprises deciding on a dose of the crop efficiency product to be applied for each of the plurality locations.

This may reduce the requirements for the crop efficiency product and also the costs. In addition, deciding on a dose for each of the plurality locations may allow a precise control of the crop health and the resulting yield.

According to an embodiment of the invention, the dose of the crop efficiency product is decided based on at least one of the following factors at each of the plurality of locations: a leaf area index, a biomass, and a stress level.

According to an embodiment of the invention, controlling at least one treatment device to comply with the decision is based on a generation of an application map indicative of the decision, for each of the plurality of locations, whether to treat or not and a delivery of the application map to the at least one treatment device. Alternatively or additionally, controlling at least one treatment device to comply with the decision is based on an algorithm embedded on the at least one treatment device adapted for being run in real time for the location the at least one treatment device passes.

The application map may comprises a plurality of grids in form of a rectangular array of squares or rectangles of equal size with an indication of whether to apply the crop efficiency product at respective grids. Preferably, the application map may also include the dose to be applied at respective locations. The application map may be marked with global positioning system (GPS) coordinates for guiding ground robots or aerial sprayers to apply the crop efficiency product at correct locations. The treatment device, e.g. ground robots with variable-rate applicator or aerial sprayers, may receive an application map before applying the crop efficiency product, so that the treatment device can be guided with the GPS coordinates of the target areas to apply the crop efficiency product. This may allow the treatment device to apply the crop efficiency products to target locations, which may have a positive return on investment.

A second aspect of the invention relates to a decision-support system for controlling a treatment device for applying a crop efficiency product to at least one crop in a field. The decision-support system comprises a data interface, a parameter determination unit, a yield prediction unit, a decision unit, a controlling unit, and a treatment control interface. The parameter determination unit is configured to determine, based on remotely-sensed data received from the data interface, at least one soil parameter at a plurality of locations in the field. The yield prediction unit is configured to generate, for each of the plurality of locations, a predicted yield response to the application of the crop efficiency product for the at least one crop based on the at least one determined soil parameter and a prediction model. The prediction model is parametrized or trained based on a sample set including a plurality of different values of the at least one soil parameter and associated yield responses for the at least one crop under the application of the crop efficiency product. The decision unit is configured to decide, for each of the plurality of locations in the field, whether to treat or not based on the predicted yield response. The controlling unit is configured to generate a treatment control signal indicative of the decision and to output the treatment control signal to the treatment control interface, which when transmitted causes an activation of at least one treatment device to comply with the decision.

For the decision-support system the same explanations apply as for the method as outlined above. The decision-support system may be used to derive soil parameters and optional vegetation parameters and to generate a predicted yield response to an application of the crop efficiency product. The predicted yield map for a plurality of locations in the field may offer a robust basis for farmers to prepare schedules for applying the crop efficiency product. For example, only locations with a positive predicted yield response above a reference value may be marked for the application of the crop efficiency product. In this way, a positive return on investment may be achieved, as the positive effect of the crop efficiency product in these locations outpaces the penalty. This may not only improve the potential yields, but also reduce the requirements for the crop efficiency product and also the costs.

The term “decision-support system” as used herein may denote a computing device or a computing system, regardless of the platform, being suitable for executing program code related to the proposed method. For example, the decision-support system may be a remote server that provides a web service to facilitate management of a plantation field e.g. by a farmer of the plantation field. The remote server may have a more powerful computing power to provide the service to multiple users to manage many different plantation fields. The remote server may include an interface through which a user can authenticate (e.g. by providing a username and password); and an interface for creating, modifying, and deleting configuration data of one or more treatment devices in the plantation fields. The configuration data may be generated by the decision-system by analyzing the remotely-sensed data. For example, the configuration data may comprise the decision including geographical information of the areas to be treated and an optimal dose to be applied to these areas. The configuration data may be loaded onto the treatment devices e.g. via a network to enable the treatment devices to perform treatment. The parameter determination unit, the yield prediction unit, the decision unit, and the controlling unit may be different data processing elements such as microprocessor, microcontroller, field programmable gate array (FPGA), central processing unit (CPU), digital signal processor (DSP) capable of receiving data, e.g. via a universal service bus (USB), a physical cable, Bluetooth, or another form of data connection. Alternatively, they may be integrated e.g. in a personal computer for providing the decision and controlling the treatment devices.

According to an embodiment of the invention, the parameter determination unit is further configured to determine, based on the collected remotely-sensed data, at least one vegetation parameter, preferably measured at a sub-field level resolution. The yield prediction unit is configured to generate, for each of the plurality of locations, a predicted yield response to the application of the crop efficiency product for the at least one crop based on the at least one determined soil parameter, the at least one determined vegetation parameter, and a prediction model, wherein the prediction model is parametrized or trained based on a sample set including a plurality of different values of the at least one soil parameter, different values of the at least one vegetation parameter, and associated yield responses for the at least one crop under the application of the crop efficiency product.

In other words, the decision-support system may consider the vegetation parameter for improving the accuracy of yield response prediction.

According to an embodiment of the invention, the decision unit is further configured to decide on a dose of the crop efficiency product to be applied for each of the plurality of locations.

The dose may allow the achievement of desirable yields for each of the plurality of locations and thus a derisible yield for the entire field.

A third aspect of the invention relates to a treatment device for applying a crop efficiency product to at least one crop in a field. The treatment device comprises a treatment control interface, a treatment controlling unit, and a treatment arrangement with one or a plurality of treatment units. The treatment control interface of the treatment device is connectable to the treatment control interface of the decision-support system to receive a treatment control signal. The treatment controlling unit is configured to regulate respective ones of treatment units of the treatment arrangement to apply the crop efficiency product at respective locations based on the received treatment control signal.

The treatment device may denote a device for applying a crop efficiency product, which may include common sprayers, ground robots with variable-rate applicators, aerial sprayers, or other variable-rate herbicide applicators. If the treatment device is a ground robot with variable-rate applicator or an aerial sprayer, the treatment device may be a GPS-guided treatment device, such as a GPS-guided ground robot or a GPS-guided aerial sprayer. The decision-support system may provide GPS coordinates for guiding the treatment devices to apply the crop efficiency products at the desirable locations, where a positive return is expected.

According to an embodiment of the invention, the treatment controlling unit is configured to run an algorithm embedded on the treatment controlling device in real time for a location the treatment device passes based on the treatment control signal.

In other words, no application map of the crop efficiency product is required. Instead, the treatment device determines whether to treat or not in real time for each location the treatment passes. This may reduce the memory requirements of the treatment device for storing the entire application map.

A fourth aspect of the invention relates to a system for applying a crop efficiency product to at least one crop in a field. The system comprises a remote sensing device, a decision-support system as described above and below, and at least one treatment device described above and below. The remote sensing device is configured to collect remotely-sensed data of the field. The decision-support system is configured to decide, based on the collected remotely-sensed data of the field, whether to treat or not, and preferably, to decide on a dose of the crop efficiency product to be applied for each of a plurality of locations in the field. The at least one treatment device is configured to be controlled by the decision-support system to comply with the decision.

The system may advantageously allow the application of the crop efficiency product ranging from mission planning, acquiring remotely-field data of a field, retrieving soil and vegetation parameters, predicting yield responses for a plurality of locations, locating areas where a positive return is expected, to practicing precision crop efficiency product application. Thus, a better return on investment with less consummation of the crop efficiency product may be achieved.

### BRIEF DESCRIPTION OF THE DRAWINGS

Exemplary embodiments of the invention will be described in the following with reference to the following drawings:

FIG. 1 shows a schematic drawing of a method according to an exemplary embodiment of the present disclosure.

FIG. 2 shows a schematic drawing of a field according to an exemplary embodiment of the present disclosure.

FIG. 3 shows a schematic drawing of a decision-support system according to an exemplary embodiment of the present disclosure.

FIG. 4 shows a schematic drawing of a treatment device according to an exemplary embodiment of the present disclosure.

FIG. 5 shows a schematic drawing of a system according to an exemplary embodiment of the present disclosure.

### DETAILED DESCRIPTION OF DRAWINGS

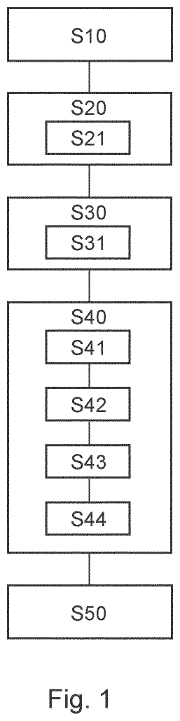

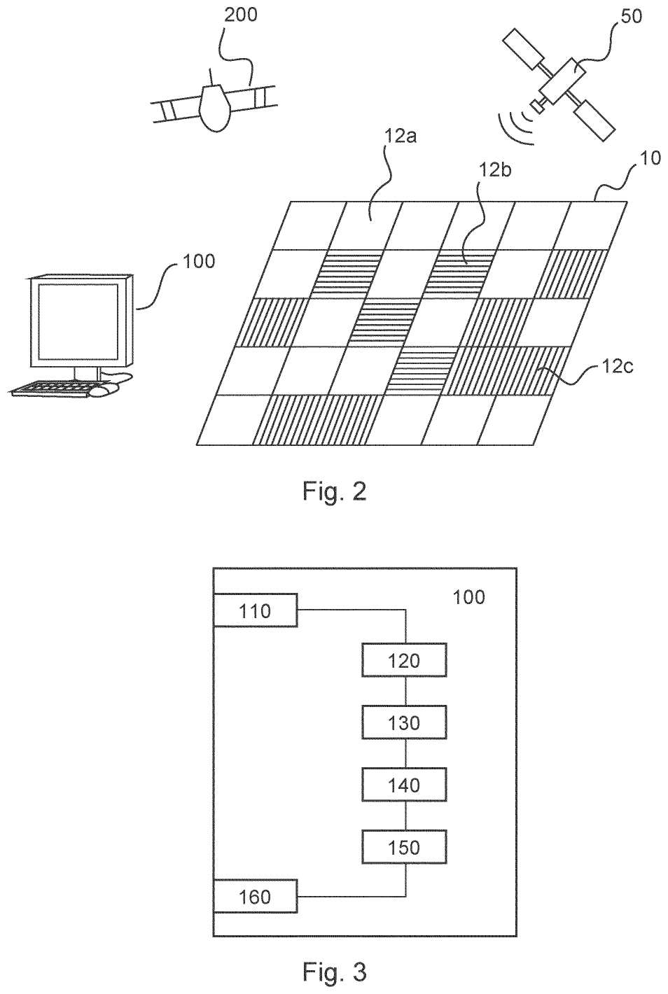

FIG. 1 shows a block diagram of an embodiment of a computer-implemented method for applying a crop efficiency product in a field 10. An example of the field 10 is illustrated in FIG. 2. In step S10, remotely-sensed data of the field may be collected before an application of the crop efficiency product in the field. The remotely-sensed data may be collected using satellite, drone, or radar platforms. To collect the remotely-sensed data, drones may be fitted with visual, IR, NIR, and/or thermal sensors. In another example, passive or active remote sensing of radar rays may be used to collect remotely-sensed data.

In step S20, at least one soil parameter at a plurality of locations in the field is determined based on the collected remotely-sensed data. For example, as illustrated in FIG. 2, the field 10 is divided into a plurality of grids in form of a rectangular array of squares 12a, 12b, 12c of equal size. The at least one soil parameter may be determined at the plurality of locations, e.g. at the plurality of squares 12a, 12b, 12c.

The soil parameter may include e.g. pH, electrical conductivity, texture, moisture, temperature, soil organic matter, available nitrogen, phosphorus and/or potassium. The soil parameters can be determined by several methods. For example, passive or active remote sensing of radar rays reflected in the soil can be used to estimate close to surface moisture, e.g. 3 to 10 cm, and surface temperature of the soil and crop. In another example, drones may be fitted with an IR camera for detecting heat signatures of soils, which allows obtaining a map depicting soil heat and moisture variations.

Preferably, the at least one soil parameter may comprise a soil moisture. Preferably, the soil moisture may be measured at sub-field resolution in a timeframe in days before the application of the crop efficiency product. Crop efficiency products may influence the crops reaction and memory to drought stress later in season (greening effect). Soil water content does indicate how much water- and heat stress a plant suffers. The soil moisture is preferably measured at a sub-field resolution of around 100 m.

Preferably, the at least one soil parameter may comprise a soil surface temperature. Preferably, the soil surface temperature may be measured during a particular time period, preferably during winter, e.g. in February and March. Winter conditions define the survival rate of spores from previous season, leading to a better yield response if more spores have survived winter. It is believed that spore survival rate correlates with soil moisture and land temperature during winter. Additionally or alternatively, the soil surface temperature may be predicted by whether forecast data. In this way, it is not required to perform the in-season measurements.

In step S30, it is generated, for each of the plurality of locations, a predicted yield response to the application of the crop efficiency product for the at least one crop based on the at least one determined soil parameter and a prediction model. The prediction model is parametrized or trained based on a sample set including a plurality of different values of the at least one soil parameter and associated yield responses for the at least one crop under the application of the crop efficiency product. For example, as illustrated in FIG. 2, a predicted yield response may be calculated for each squares 12a, 12b, 12c. The plain squares 12a may denote locations with negative predicted yield responses. The patterned squares 12b may denote locations with predicted yield responses of a low positive value. The patterned squares 12 c may denote locations with predicted yield responses of a positive value above a reference value.

In other words, previous data and current measurements of soil parameters may serve in yield forecasting. In an example, the yield prediction model is a machine learning model. Machine learning algorithms build a mathematical model of training data from field trials, in order to make predictions or decisions based on the at least one determined soil parameter without being explicitly programmed to perform the task. In another example, the yield prediction model comprises a mathematical equation for correlating the predicted yield response with the at least one determined soil parameter.

In step S40, it is decided, for each of the plurality of locations in the field, whether to treat or not based on the predicted yield response. Information indicative of the decision is outputted useable to activate at least one treatment device to comply with the decision. For example, a positive predicted yield response at a location may indicate that the location is worth to be treated, whereas a negative predicted yield response at a location may indicate that the location is not worth to be treated. For example, as illustrated in FIG. 2, the patterned squares 12b and 12c have a positive yield response and thus may be worth to be treated. On the other hand, the plain squares 12a may have a negative yield response and thus may not be worth to be treated. In optional step S41, it is evaluated, based on the predicted yield response, whether a treatment i) deteriorates a growth of the at least one crop, ii) does not affect the growth of the at least one crop, or iii) improves the growth of the at least one crop. For example, as illustrated in FIG. 2, the patterned squares 12b and 12c have a positive yield response and thus improve the growth of the at least one crop. On the other hand, the plain squares 12a have a negative yield response and thus may deteriorate the growth of the at least one crop.

In optional step S42, it is determined, for each of the plurality of locations, whether the predicted yield response is above a positive reference value. As discussed above, the patterned squares 12b and 12c have a positive yield response and thus improve the growth of the at least one crop. However, the patterned squares 12b denote locations with predicted yield responses of a low positive value. In other words, although positive yield responses can be seen for these locations, the positive return on the investment are relatively low. Thus, it may not be reasonable to apply the crop efficiency product to these locations. On the other hand, the patterned squares 12c denote locations with predicted yield responses of a positive value above a reference value. Thus, a more positive return on investment can be seen for these locations. It may be reasonable to apply the crop efficiency product to the locations denoted with patterned squares 12c.

In optional step S43, it is decided, for each of the plurality of locations, whether to treat or not based on the determination result. Thus, the crop efficiency product is applied to the locations denoted with patterned squares 12c.

In optional step S50, at least one treatment device is controlled to comply with the decision based on the outputted information. The at least one treatment device may include a common sprayer or a crop duster, such as an airplane spraying chemicals. For example, the at least one treatment device may be controlled to apply the crop efficiency product only at locations denoted with patterned squares 12c.

Controlling at least one treatment device to comply with the decision may be conducted based on a generation of an application map indicative of the decision, for each of the plurality of locations, whether it is worth to treat or not, and a delivery of the application map to the at least one treatment device. For example, as illustrated in FIG. 2, each square 12a, 12b, 12c in the field 10 may be provided with a GPS coordinate. The squares 12a, 12b, 12c and the corresponding GPS coordinates may form an application map, which can guide GPS-guided ground robots or GPS-guided aerial sprayers to apply the crop efficiency product at the desired locations, e.g. locations denoted with patterned squares 12c. Alternatively or additionally, an algorithm embedded on the at least one treatment device may be run in real time for the location the at least one treatment device passes such that the at least one treatment device is controlled to comply with the decision.

Optionally, determining S20 at least one soil parameter at a plurality of locations in the field further comprises the step of determining S21, based on the collected remotely-sensed data, at least one vegetation parameter, preferably measured at a sub-field level resolution. The vegetation parameter may comprise SPI, VOD, NDVI, and/or EVI. The vegetation parameter may be obtained by analysing the spectral signatures of the crop and soil in the image data collected using optical remote sensing techniques. Generating S30 a predicted yield response to the application of the crop efficiency product further comprises the step of generating S31, for each of the plurality of locations, a predicted yield response to the application of the crop efficiency product for the at least one crop based on the at least one determined soil parameter, the at least one vegetation parameter, and a prediction model. The prediction model is parametrized or trained based on a sample set including a plurality of different values of the at least one soil parameter, different values of the at least one vegetation parameter, and associated yield responses for the at least one crop under the application of the crop efficiency product. In other words, an additional parameter, i.e. at least one vegetation parameter, may be provided as a further parameter input for the prediction model, such as a machine learning model or a mathematical equation. This additional parameter may increase the accuracy in predicting the yield response to the application of the crop efficiency product.

Optionally, deciding S40, for each of the plurality of locations, whether to treat or not, further comprises the step of deciding S44 on a dose of the crop efficiency product to be applied for each of the plurality locations. The dose of the crop efficiency product is decided based on at least one of the following factors at each of the plurality of locations: a leaf area index, a biomass, and a stress level. For example, lower biomass zones may be applied with the crop efficiency product with a lower dose. For example, if a non-linear yield response to these factors is assumed, a lower dose of the crop efficiency product may be applied to a lower stress zone, whereas a higher dose may be applied to a higher stress zone.

FIG. 3 schematically shows an embodiment of a decision-support system 100 for controlling a treatment device for applying a crop efficiency product in a field. An example of the decision-support system 100 in form of a computer system is illustrated in FIG. 2. The decision-support system 100 may be a remote server that provides a remote service e.g. via internet, to facilitate upload and management of remotely-sensed data from many different plantation fields collected by the farmers. The remote server may include an interface through which a user (e.g. a farmer) can manage the remotely-sensed data and related information. For example, the decision-support system 100 may interface with users with webpages served by the decision-support system to facilitate the management of the remotely-sensed data and related decisions. The related decision may include e.g. one or more target areas to be treated, an optimum dose of the crop efficiency product to be applied for these areas, etc. The related decision may be part of configuration data, which may be loaded onto the one or more treatment devices in the plantation field e.g. via a network, to enable the one or more treatment devices to perform a treatment on the target areas. Alternatively, the decision-support system 100 may be a local computing device, such as a personal computer (PC).

The decision-support system 100 comprises a data interface 110, a parameter determination unit 120, a yield prediction unit 130, a decision unit 140, a controlling unit 150, and a treatment control interface 160.

The data interface 110 may be a secure digital (SD) memory card interface, a universal serial bus (USB) interface, a Bluetooth interface, a wireless network interface, etc. suitable to receive the remotely-data collected using satellite, radar or drone platforms. The remotely-sensed data may comprise radar image data or optical image data. The remotely-sensed data may also comprise GPS data adapted for providing guidance of the treatment devices to the target areas.

The parameter determination unit 120 is configured to determine, based on remotely-sensed data received from the data interface, at least one soil parameter at a plurality of locations in the field. The at least one soil parameter may comprise a soil temperature and/or a soil moisture. A variety of remote sensing techniques for soil moisture retrieval may be used based on their different electromagnetic spectrum. In an example, if active remote sensing of radar rays is used, the soil moisture or soil surface temperature may be determined from the remotely-sensed data based on backscatter coefficient and dielectric properties. In another example, if visible sensors are used, soil moisture and soil surface temperature may be determined from the remotely-sensed data based on soil albedo index of refraction. In a further example, if thermal infrared sensors are used, soil moisture may be determined from the remotely-sensed data by measuring soil surface temperature.

Optionally, the parameter determination unit 120 is further configured to determine, from the received remotely-sensed data, at least one vegetation parameter, such as SPI, VOD, NDVI, and/or EVI, preferably measured at a sub-field level resolution.

The yield response unit 130 is configured to generate, at each of the plurality of locations, a predicted yield response to the application of the crop efficiency product for the at least one crop based on the at least one determined soil parameter and a prediction model. The prediction model is parametrized or trained based on a sample set including a plurality of different values of the at least one soil parameter and associated yield responses for the at least one crop under the application of the crop efficiency product. In an example, the yield prediction model is a machine learning model using training data from field trials. In another example, the yield prediction model is a mathematical equation for correlating the predicted yield response with the at least one soil parameter. Optionally, the yield prediction unit 130 is configured to generate, at each of the plurality of locations, a predicted yield response to the application of the crop efficiency product for the at least one crop based on the at least one determined soil parameter, the at least one determined vegetation parameter, and a prediction model. The prediction model is parametrized or trained based on a sample set including a plurality of different values of the at least one soil parameter, different values of the at least one vegetation parameter, and associated yield responses for the at least one crop under the application of the crop efficiency product.

The decision unit 140 is configured to decide, for each of the plurality of locations in the field, whether to treat or not based on the predicted yield response. Optionally, the decision unit 450 is further configured to decide on a dose of the crop efficiency product to be applied for each of the plurality of locations.

The controlling unit 150 is configured to generate a treatment control signal indicative of the decision and to output the treatment control signal to the treatment control interface 160, which when transmitted causes an activation of at least one treatment device to comply with the decision.

Thus, the parameter determination unit 120, the yield response unit 130, the decision unit 140, and the controlling unit 150 may be part of, or include a general-purpose processing unit, a graphics processing unit (GPU), a microcontroller and/or microprocessor, a field programmable gate array (FPGA), a digital signal processor (DSP), and equivalent circuitry, alone or in combination. Furthermore, the above-described units may be connected to volatile or non-volatile storage, display interfaces, communication interfaces and the like as known to a person skilled in the art.

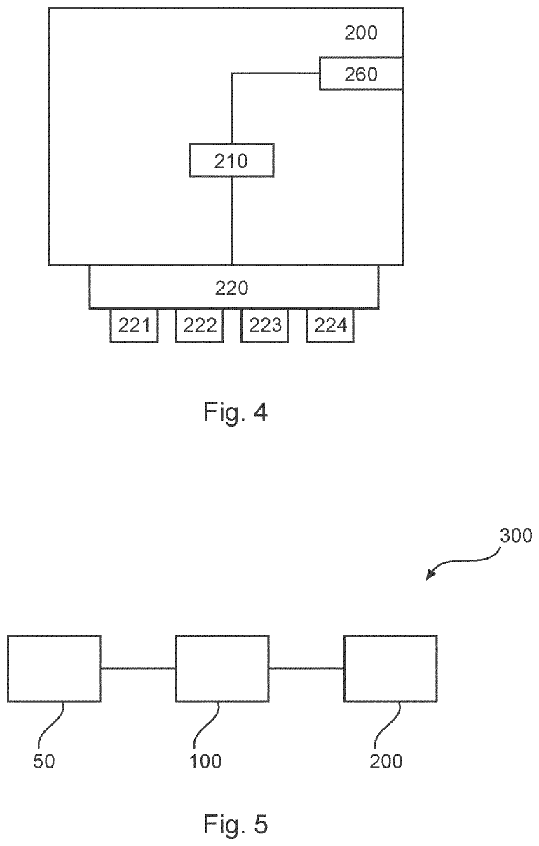

FIG. 4 schematically shows an embodiment of a treatment device 200 for applying a crop efficiency product in a field. The treatment device 200 comprises a treatment control interface 260, a treatment controlling unit 210, and a treatment arrangement 220 with one or a plurality of treatment units 221, 222, 223, 224.

The treatment device 200 may be e.g. ground robots with variable-rate applicators, aerial sprayers, or other variable-rate herbicide applicators. The treatment device 200 may also be a common sprayer. An example of the treatment device 200 in form of a crop duster is illustrated in FIG. 2. The treatment arrangement 220 may be a nozzle arrangement comprising a plurality of nozzles as treatment unit 221, 222, 223, 224.

The treatment control interface 260 of the treatment device is connectable to the treatment control interface 160 of the decision-support system 100 as discussed in FIG. 3. This may be done wirelessly, thus allowing a remote control of the treatment device 200 via the decision-support system 100. The treatment control interface 260 is configured to receive a treatment control signal indicative of the decision, for each of the plurality of locations, whether to treat or not. Optionally, the decision may include a dose to be applied for each of the plurality locations.

The treatment controlling unit 210 is configured to regulate respective ones of treatment units 221, 222, 223, 224 of the treatment arrangement 220 to apply a crop efficiency product to respective locations based on the received treatment control signal. Optionally, the treatment controlling unit 210 is configured to run an algorithm embedded on the treatment controlling device in real time for a location the treatment device passes based on the treatment control signal.

FIG. 5 schematically shows an embodiment of a system 300 for applying a crop efficiency product in a field. The system comprises a remote sensing device 50, a decision-support system 100 as described above and at least one treatment device 200 as described above. The remote sensing device 50, the decision-support system 100 and the at least one treatment device 200 may be associated with a network. For example, the network may be the internet. The network may alternatively be any other type and number of networks. For example, the network may be implemented by several local area networks connected to a wide area network. The network may comprise any combination of wired networks, wireless networks, wide area networks, local area networks, etc. In some implementations, the decision-support system 100 may be a server to provide a web service to facilitate management of a plantation field. The user (e.g. a farmer) may collect remotely-sensed data with a drone in his plantation field. He may uploaded the remotely-sensed data e.g. via the network to the decision-support system 100 for further analysis. The decision-support system 100 may output a treatment control signal comprising the configuration information of the treatment devices for activating these treatment devices to comply with the decision.

The remote sensing device 50 is configured to collect remotely-sensed data of a field. The remote sensing device 50 may be e.g. a satellite, a radar, or a drone. An example of the remote sensing device 50 in form of a satellite is illustrated in FIG. 2. Optical remote sensing may be carried out to make use of e.g. visible, IR, NIR or multispectral sensors to form images of surface of the field by detecting the solar radiation reflected from targets on the ground. Satellite sensors or radars operating at microwaves, both active and passive, for the remote sensing monitoring of surface of the field.

The decision-support system 100 is configured to decide, based on the collected remotely-sensed data of the field, whether to treat or not, and preferably, to decide on a dose of the crop efficiency product to be applied for each of a plurality of locations in the field.

The treatment device 200 is configured to be controlled by the decision-support system to comply with the decision.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

### REFERENCE LIST

10 Field
12a location in the field
12b location in the field
12c location in the field
50 remote sensing device
100 decision-support system
110 data interface
120 parameter determination unit
130 yield prediction unit
140 decision unit
150 controlling unit
160 treatment control interface
200 treatment device
210 treatment controlling
220 treatment arrangement
221 treatment unit
222 treatment unit
223 treatment unit
224 treatment unit
260 treatment control interface
300 system
S10 collecting remotely-sensed data
S20 determining at least one soil parameter
S21 determining at least one vegetation parameter
S30 generating a predicted yield response
S31 generating a predicted yield response
S40 deciding whether to treat
S41 evaluating whether a treat deteriorates, does not affect or improves the growth
S42 determining whether the predicted yield response is above a positive reference value
S43 deciding whether to treat
S44 deciding on a dose
S50 controlling at least one treatment device

1. A computer-implemented method for applying a crop efficiency product to at least one crop in a field, the method comprising:
collecting (S10), by a data interface (110), remotely-sensed data of the field before an application of the crop efficiency product in the field;
determining (S20), by a parameter determination unit (120), based on the collected remotely-sensed data, at least one soil parameter at a plurality of locations in the field;
generating (S30), by a yield prediction unit (130), for each of the plurality of locations, a predicted yield response to the application of the crop efficiency product for the at least one crop based on the at least one determined soil parameter and a prediction model, wherein the prediction model is parametrized or trained based on a sample set including a plurality of different values of the at least one soil parameter and associated yield responses for the at least one crop under the application of the crop efficiency product; and
deciding (S40), by a decision unit (140), for each of the plurality of locations in the field, whether to treat or not based on the predicted yield response, and outputting information indicative of the decision useable to activate at least one treatment device to comply with the decision.

2. The method according to claim 1, further comprising:
controlling (S50), by a controlling unit (150), at least one treatment device to comply with the decision based on the outputted information.

3. The method according to claim 1,
wherein the at least one soil parameter comprises at least one of the following:
a soil moisture, preferably measured at a sub-field resolution in a timeframe in days before the application of the crop efficiency product; and/or
a soil surface temperature, preferably measured during a particular time period, preferably during winter.

4. The method according to claim 3,
wherein the soil surface temperature is predicted by weather forecast data.

5. The method according to claim 1,
wherein determining (S20) at least one soil parameter at a plurality of locations in the field further comprises:
determining (S21), based on the collected remotely-sensed data, at least one vegetation parameter, preferably measured at a sub-field level resolution; and
wherein generating (S30) a predicted yield response to the application of the crop efficiency product further comprises:
generating (S31), for each of the plurality of locations, a predicted yield response to the application of the crop efficiency product for the at least one crop based on the at least one determined soil parameter, the at least one vegetation parameter, and a prediction model, wherein the prediction model is parametrized or trained based on a sample set including a plurality of different values of the at least one soil parameter, different values of the at least one vegetation parameter, and associated yield responses for the at least one crop under the application of the crop efficiency product.

6. The method according to claim 1,
wherein deciding (S40), for each of the plurality of locations, whether to treat or not, further comprises:
evaluating (S41), based on the predicted yield response, whether a treatment i) deteriorates a growth of the at least one crop, ii) does not affect the growth of the at least one crop, or iii) improves the growth of the at least one crop;
determining (S42), for each of the plurality of locations, whether the predicted yield response is above a positive reference value; and
deciding (S43), for each of the plurality of locations, whether to treat or not based on the determination result.

7. The method according to claim 1,
wherein deciding (S40), for each of the plurality of locations, whether to treat or not, further comprises:
deciding (S44) on a dose of the crop efficiency product to be applied for each of the plurality locations.

8. The method according to claim 7,
wherein the dose of the crop efficiency product is decided based on at least one of the following factors at each of the plurality of locations:
a leaf area index;
a biomass; or
a stress level.

9. The method according to claim 1,
wherein controlling (S50) at least one treatment device to comply with the decision is conducted based on:
i) a generation of an application map indicative of the decision, for each of the plurality of locations, whether to treat or not, and a delivery of the application map to the at least one treatment device; and/or
ii) an algorithm embedded on the at least one treatment device adapted for being run in real time for the location the at least one treatment device passes.

10. A decision-support (100) system for controlling a treatment device for applying a crop efficiency product to at least one crop in a field, the decision-support system comprising:
a data interface (110);
a parameter determination unit (120);
a yield prediction unit (130);
a decision unit (140);
a controlling unit (150); and
a treatment control interface (160);
wherein the parameter determination unit is configured to determine, from remotely-sensed data received from the data interface, at least one soil parameter at a plurality of locations in the field;
wherein the yield prediction unit is configured to generate, at each of the plurality of locations, a predicted yield response to the application of the crop efficiency product for the at least one crop based on the at least one determined soil parameter and a prediction model, wherein the prediction model is parametrized or trained based on a sample set including a plurality of different values of the at least one soil parameter and associated yield responses for the at least one crop under the application of the crop efficiency product;
wherein the decision unit is configured to decide, for each of the plurality of locations in the field, whether to treat or not based on the predicted yield response; and
wherein the controlling unit is configured to generate a treatment control signal comprising information indicative of the decision and to output the treatment control signal to the treatment control interface, which when transmitted causes an activation of at least one treatment device to comply with the decision.

11. The decision-support system according to claim 10,
wherein the parameter determination unit is further configured to determine, from the received remotely-sensed data, at least one vegetation parameter, preferably measured at a sub-field level resolution; and
wherein the yield prediction unit is configured to generate, at each of the plurality of locations, a predicted yield response to the application of the crop efficiency product for the at least one crop based on the at least one determined soil parameter, the at least one determined vegetation parameter, and a prediction model, wherein the prediction model is parametrized or trained based on a sample set including a plurality of different values of the at least one soil parameter, different values of the at least one vegetation parameter, and associated yield responses for the at least one crop under the application of the crop efficiency product.

12. The decision-support system according to claim 10,
wherein the decision unit is further configured to decide on a dose of the crop efficiency product to be applied for each of the plurality of locations.

13. A treatment device (200) for applying a crop efficiency product to at least one crop in a field, comprising:
a treatment control interface (260);
a treatment controlling unit (210); and
a treatment arrangement (220) with one or a plurality of treatment units (221, 222, 223, 224);
wherein the treatment control interface of the treatment device is connectable to the treatment control interface of the decision-support system according to claim 10 to receive a treatment control signal; and
wherein the treatment controlling unit is configured to regulate respective ones of treatment units of the treatment arrangement to apply the crop efficiency product at respective locations based on the received treatment control signal.

14. The treatment device according to claim 13,
wherein the treatment controlling unit is configured to run an algorithm embedded on the treatment controlling device in real time for a location the treatment device passes based on the treatment control signal.

15. A system (300) for applying a crop efficiency product to at least one crop in a field, comprising:
a remote sensing device (50);
a decision-support system according to claim 10; and
at least one treatment device;
wherein the remote sensing device is configured to collect remotely-sensed data of the field;
wherein the decision-support system is configured to decide, based on the collected remotely-sensed data of the field, whether to treat or not, and preferably, to decide on a dose of the crop efficiency product to be applied for each of a plurality of locations in the field; and
wherein the at least one treatment device is configured to be controlled by the decision-support system to comply with the decision.
